(19) United States
(12) Patent Application Publication
(10) Pub. No.: US 2026/0047897 A1
(43) Pub. Date: Feb. 19, 2026

(54) TECHNICAL AND THEORETICAL SPECIFICATIONS FOR SMALL-SCALE MACHINES AND MICRO-ROBOTS FOR DESTROYING ANY HARMFUL MATTER INSIDE A LIVING ORGANISM USING A SUPERCRITICAL FLUID

(71) Applicant: Jason Hoffman, Atlanta, GA (US)

(72) Inventor: Jason Hoffman, Atlanta, GA (US)

(21) Appl. No.: 18/805,528

(22) Filed: Aug. 14, 2024

(51) Int. Cl. A61B 34/30; A61B 17/00

(52) U.S. Cl. CPC A61B 34/30; A61B 17/00234; A61B 2017/00345

(57) ABSTRACT

Any and all nano-, micro-, or small-scale robots, devices, machines, etc. in general that may or can be used inside any living organism, such as the human body or animals, to specifically administer supercritical fluids of any type and any dose size, especially supercritical water, to destroy, eliminate, etc. harmful or undesired matter or substances, including but not limited to per-and polyfluoroalkyl substances, small-scale synthetic chemicals in general, and plastics such as micro-and nanoplastics. Any nano-and micro-sizes refer to all nanometers and all micrometers. Small-scale increases the size to millimeters where some of these said devices may operate but less likely so, especially in relation to micrometers and nanometers. In current technology, many of these devices operate in the tens of micrometers, such as a 20-micrometer robot for biotechnology and engineering. A living organism may be living anywhere or everywhere.

### TECHNICAL FIELD

This disclosure relates generally to any and all nano-, micro-, or small-scale robots, devices, machines, etc. and their variations and complexities that may or can be used inside any living organism, such as the human body or animals, to specifically administer supercritical fluids of any type and any dose size, especially supercritical water, to destroy, eliminate, etc. harmful or undesired matter or substances, including but not limited to per-and polyfluoroalkyl substances, small-scale synthetic chemicals in general, and plastics such as micro-and nanoplastics. Any nano-and micro-sizes refer to all nanometers and all micrometers. By writing small-scale, the inventor increases the size from nanometers to micrometers and finally to millimeters where some of these said devices may operate but less likely so, especially in relation to micrometers and nanometers. In current technology, many of these devices operate in the tens of micrometers, such as a 20-micrometer robot for biotechnology and engineering. Additionally, a living organism may be living anywhere or everywhere.

### BACKGROUND

The present invention relates to novel technical and theoretical specifications for creating any and all nano-, micro-, or small-scale robots, devices, machines, etc. and their variations and complexities that may or can be used inside any living organism, such as the human body or animals, to specifically administer supercritical fluids of any type and any dose size, especially supercritical water, to destroy, eliminate, etc. harmful or undesired matter or substances, including but not limited to per-and polyfluoroalkyl substances, small-scale synthetic chemicals in general, and plastics such as micro-and nanoplastics. Any nano-and micro-sizes refer to all nanometers and all micrometers. By writing small-scale, the inventor increases the size from nanometers to micrometers and finally to millimeters where some of these said devices may operate but less likely so, especially in relation to micrometers and nanometers. In current technology, many of these devices operate in the tens of micrometers, such as a 20-micrometer robot for biotechnology and engineering. Additionally, a living organism may be living anywhere or everywhere.

Mankind's need to destroy harmful matter and substances, especially per- and polyfluoroalkyl (PFAS) substances, commonly known as “forever chemicals,” small-scale synthetic chemicals in general, and plastics such as micro-and nanoplastics, are nearly universally recognized and well-documented. PFAS and harmful synthetic chemicals in general are nearly ubiquitous and dangerous to all living organisms everywhere. American medical institutions and health organizations claim PFAS may be linked to countless health problems and deterioration, from causing various cancers to drastically reducing human fertility rates. In the journal “Science of The Total Environment,” a paper was published this year (2024) titled “Fluorine mass balance analysis in wild boar organs from the Bohemian Forest National Park,” by Till Schröder, Viktoria Müller, Marc Preihs, Jan Borovic̆ka, Raquel Gonzalez de Vega, Andrew Kindness, and Jörg Feldmann, claiming wild boar in a European national park, Bohemian Forest National Park, have been found to contain levels of toxic PFAS nearly five-times higher than is allowed to be sold in meat for human consumption under EU law. Yet “in this study wild boars were obtained from the Bohemian Forest NP (Czech Republic), where all river systems originate from inside the park. Therefore, PFAS deposition can mainly occur via atmospheric transport or from the limited human activity inside the NP, making it an ideal place to study PFAS contamination in wildlife for background contamination. We analysed kidney and liver of wild boar (from both gender and different age) for targeted PFAS, suspect screening, EOF, and dTOPA, to gain an understanding of legacy and precursor PFAS in the environment as background contamination via atmospheric transport in central Europe.” This makes it seems like PFAS and harmful synthetic chemicals in general are nearly ubiquitous and dangerous to all living organisms everywhere, regardless of whether they be on Earth or off the planet, such as on the International Space Station (ISS), Mars (in the future), or anywhere else in the universe or multiverse

A supercritical fluid (SCF) refers to a fluid that is at a state above its critical point. For instance, one of the most common types of fluids used in SCF technology is water. Supercritical water (SCW), which refers to water that is in a fluid state above its critical point, occurs at temperatures above 374° C. (or 705° F.) and pressures above 22.1 megapascals (MPa or 3,212 psi). In this state, water exhibits properties of both a liquid and a gas, with no distinct phase boundary, which apply more generally to SCF. SCF and SCW have unique characteristics, including: 1) High diffusivity and low viscosity (gas-like properties), 2) High solvation power (liquid-like properties), 3) Ability to dissolve organic compounds and gases, 4) Lower dielectric constant compared to liquid water, leading to solubility reversal. SCF, and particularly SCW, are useful in various applications, such as waste treatment and water reclamation in waste streams, chemical synthesis, geothermal energy extraction, power generation, materials processing, food processing, and destruction or elimination of harmful or undesired matter or substances, including but not limited to per-and polyfluoroalkyl substances, small-scale synthetic chemicals in general, and plastics such as micro-and nanoplastics.

Existing commercial systems are beginning to demonstrate the potential of SCF and SCW technologies in the listed applications. They are currently implemented on a large scale, often in industrial settings, including shipping containers or specialized facilities. These scale devices and systems require advanced materials, designs, among other things to sustain high reaction temperatures and pressures for containing SCF or SCW.

Additionally, at near-critical conditions, any fluid may undergo a unique change in its thermophysical properties: the solubility reversal. This means once soluble substances now become insoluble. For instance, for water soluble, such as salts, now become insoluble.

At greater than 100 nm, supercritical water exists at this scale as previously described. The volume is large enough to exhibit bulk properties, though increased surface effects may cause deviations from macroscopic behavior. Generally, nanoscale effects become significant below 100 nm, where surface area to volume ratios increase dramatically, affecting properties like surface tension and reactivity. For scales between 10-100 nm, the nature of supercritical water becomes more complex. At the larger end of this range, supercritical-like behavior might still be observed, but properties may differ significantly from bulk supercritical water due to strong surface effects and confinement. Quantum effects typically become significant below 10 nm, where quantum confinement can influence electronic and optical properties, making the concept of supercritical water less meaningful. At these scales, small clusters of water molecules dominate, with behavior governed by molecular interactions and confinement effects. Specifically, when supercritical water is confined in nanopores less than 10 nm, its properties can change significantly, affecting density fluctuations, phase behavior, and critical points. In extreme confinement (below 2-5 nm), quantum effects might influence molecular interactions and hydrogen bonding, though changes in thermophysical properties like diffusion rates, viscosity, and heat capacity are more due to classical confinement effects. Quantum effects may also alter hydrogen bonding or ionization behavior at scales approximately 1-10 nanometers, where classical molecular dynamics assumptions begin to break down. At even smaller scales, approaching the atomic level (sub-nanometer), quantum electrodynamics (QED) effects can influence light interactions with supercritical water, potentially altering optical and electronic properties. Thus, quantum effects could significantly influence the properties of supercritical water at nanoscale dimensions below 10 nanometers, but at larger scales (micrometers and above), its behavior is primarily governed by classical thermodynamics.

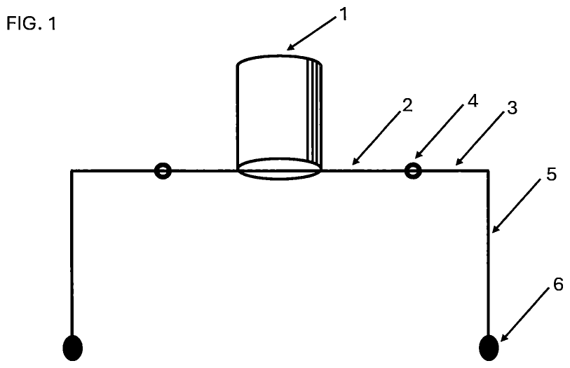

Therefore, it is important to note that at the 5-30 μm scale and dimensions of the microrobot discussed later (and as shown in FIG. 1), there would not typically be significant deviations observed from bulk supercritical water properties due to size effects. Quantum effects would be minimal to negligible at this scale for supercritical water. To observe significant changes in supercritical water properties due to size effects, the scale would generally need to go down to nanometers, with effects starting to show at below 100 nm, more pronounced as we get closer to 10 nm in size and then 5 nm, and especially so nearing 1 nm, and when we venture to sub-nanometers, QED effects are very strong.

This invention and its variations, complexities, forms, etc. sits at the interface of theoretical foundations and applied, practical ideas and can soon, in the very near future, transition fully from theoretical foundations to practical applications.

### BRIEF DESCRIPTION OF THE DRAWINGS

Features, embodiments, and advantages of the present disclosure are better understood when the following Description of the Present Invention is read with reference to the accompanying drawings.

Given the generality as well as the wide breadth and scope of the inventor's claim, FIG. 1 is an illustration of just one example of any nano-, micro-, or small-scale robot, device, machine, etc. in general that may or can be used inside any living organism, such as the human body or animals, to specifically administer supercritical fluids of any type and any dose size, especially supercritical water, to destroy, eliminate, etc. harmful or undesired matter or substances, including but not limited to per-and polyfluoroalkyl substances, small-scale synthetic chemicals in general, and plastics such as micro-and nanoplastics, according to aspects of the present disclosure. Recall for required consistency by the patent office, any nano-and micro-sizes refer to all nanometers and all micrometers. By writing small-scale, the inventor increases the size from nanometers to micrometers and finally to millimeters where some of these said devices may operate but less likely so, especially in relation to micrometers and nanometers. In current technology, many of these devices operate in the tens of micrometers, such as a 20-micrometer robot for biotechnology and engineering. Additionally, a living organism may be living anywhere or everywhere.

### DESCRIPTION OF THE PRESENT INVENTION

A description herein will describe FIG. 1, which illustrates one form of any and all nano-, micro-, or small-scale robots, devices, machines, etc. and their variations and complexities that may or can be used inside any living organism, such as the human body or animals, to specifically administer supercritical fluids of any type and any dose size, especially supercritical water, to destroy, eliminate, etc. harmful or undesired matter or substances, including but not limited to per-and polyfluoroalkyl substances, small-scale synthetic chemicals in general, and plastics such as micro-and nanoplastics.

However, the inventor is claiming all variations, forms, and complexities of this specific application and administration of supercritical fluid of any type and any dose size. any nano-and micro-sizes refer to all nanometers and all micrometers. By writing small-scale, the inventor increases the size from nanometers to micrometers and finally to millimeters where some of these said devices may operate but less likely so, especially in relation to micrometers and nanometers. In current technology, many of these devices operate in the tens of micrometers, such as a 20-micrometer robot for biotechnology and engineering. Additionally, a living organism may be living anywhere or everywhere.

In FIG. 1, the design features a microrobot and this is the variation that will be discussed.

In FIG. 1, this microrobot has two arms 5 where each is approximately 15 micrometers (μm) in length, 1 μm in height and 0.5 μm or 500 nanometers (nm) in thickness. When parallel, each arm is separated by approximately 30 μm and is connected by a base 3 and an inner base 2. Approximately center of the inner base, the three-dimensional (3D) cylinder 1 is the apparatus used to administer the packed supercritical fluid of any type and of any dose size from nanometers to micrometers in volume or area. The two end support structures 6 are heavier than the two inner support structures 4 to counterbalance the center of gravity of this microrobot so it can travel more efficiently through a living organism.

The 3D cylinder in FIG. 1, this microrobot, is 5 micrometers (μm) in width and 10μm in length, which gives it a volume of approximately 62.5π μm cubed. These dimensions form the legend that should be used to arrive at other dimensions, including but not limited to the approximately 30 μm total base length or the approximately 15 micrometers length of each arm.

These illustrative examples are mentioned not to limit or define the disclosure, but to aid understanding thereof. Additional embodiments may be discussed next, below, further on or later on, and further description is provided there.

The prior art for this invention design originates from biophysics and biotechnology for single-cell actuation and control, such as to control cell-cell interaction that may or may not occur without microstructures and microrobot tools.

One of the unique aspects of this invention is the extension of extreme packing to any supercritical fluid inside the 3D cylinder 1 as shown in FIG. 1.

It is well known that each human cell contains approximately 2 meters of DNA if stretched end-to-end; yet the nucleus of a human cell, which contains the DNA, is only about 6 μm in diameter. This is an extreme packing ratio greater than 300,000:1 and geometrically equivalent to packing 40 km (24 miles) of extremely fine thread into a tennis ball. Though this great packing and condensing occurs in a similar manner throughout the human body, in our colons, lungs and alveoli, neuronal axon, etc., and extends to the rest of nature, such as spider silk, plant roots, and among many others. Society has been mimicking nature to repeat this in electromagnetic, hardware devices such as solenoids to advanced computing, such as software compression algorithms, and much more.

The 3D cylinder in FIG. 1, this microrobot, is 5 micrometers (μm) in width and 10 μm in length, which gives it a volume of approximately 62.5π μm cubed. Given the length of DNA is 2 meters and the thickness is 2 nanometers (nm), then the volume of DNA if modeled as a cylindrical object would be 2.5π μm cubed, using 2 nm divide by 2 as the radius and 2 meters as the length. Hence, the number of 2-meter-long DNA strands that can fit inside this cylinder is 31.25. Finally, multiply the number of DNA strands by the length of each strand and the result is 62.5 meters. So, 62.5 meters of DNA-like material can fit into a cylinder with dimensions 5 μm in diameter and 10 μmin length.

With this extremely dense packing method, a theoretical ceiling of 62.5 meters worth of supercritical fluid could be packed into this relatively tiny cylindrical object at the center of this microrobot and then be administered at tiny doses of nm or μm size to undesired or harmful matter or substances, such as PFAS, which are trapped by the microrobot.

Therefore, this invention claims the extension of extreme packing of any supercritical fluid inside any object, such as the 3D cylinder 1 as shown in FIG. 1 in this microrobot.

This invention could use techniques or methods that are not novel and demonstrated by prior art for sensing and then actuating undesired and harmful matter and substances, such as PFAS. For instance, prior art demonstrates ways of actuating cells with and or combining optical sensing with microtechnology, such as microstructures and microrobots tools, arms, etc.

Another unique aspect of this invention is the administration of supercritical fluids of any type and any dose to destroy undesired and harmful matter and substances, such as PFAS.

Returning to the novelty of the technical and theoretical specifications of this invention, the supercritical fluid doses may be of any size, but the size of the doses would have to correlate with the size of the living organism and the timing between doses, given that living organisms have ranges of acceptable percentages or levels of water, salts, ionic compounds in general, carbon dioxide, etc. that are safe within their bodies. To not exceed healthy levels of water, salts, or carbon dioxide, etc., the timing of administrating would have to be spaced out to give time for the living organism to excrete excess levels created from after the administration of small doses of supercritical fluids.

Recall from the Background section: Therefore, it is important to note that at the 5-30 μm scale and dimensions of the microrobot discussed later (and as shown in FIG. 1), there would not typically be significant deviations observed from bulk supercritical water properties due to size effects. Quantum effects would be minimal to negligible at this scale for supercritical water. To observe significant changes in supercritical water properties due to size effects, the scale would generally need to go down to nanometers, with effects starting to show at below 100 nm, more pronounced as we get closer to 10 nm in size and then 5 nm, and especially so nearing 1 nm, and when we venture to sub-nanometers, QED effects are very strong.

With extreme packing techniques, which are a part of the claim of this invention, and the advancement of stronger materials that can withstand the conditions required for containing supercritical water at smaller scales, this invention and its variations, complexities, forms, etc. sits at the interface of theoretical foundations and applied, practical ideas and can soon, in the very near future, transition fully from theoretical foundations to practical applications.

1. Any and all nano-, micro-, or small-scale robots, devices, machines, etc. in general that may or can be used inside any living organism, such as the human body or animals, to specifically administer supercritical fluids of any type and any dose size, especially supercritical water, to destroy, eliminate, etc. harmful or undesired matter or substances, including but not limited to per- and polyfluoroalkyl substances, small-scale synthetic chemicals in general, and plastics such as micro- and nanoplastics. Any nano-and micro-sizes refer to all nanometers and all micrometers. By writing small-scale, the inventor increases the size from nanometers to micrometers and finally to millimeters where some of these said devices may operate but less likely so, especially in relation to micrometers and nanometers. In current technology, many of these devices operate in the tens of micrometers, such as a 20-micrometer robot for biotechnology and engineering. Additionally, a living organism may be living anywhere or everywhere. This claim is broad, brief and general in the spirit of patented technology and art: Metoprolol Succinate (U.S. Pat. No. 5,081,154).
This claim is broad, brief and general in the spirit of patented technology and art:
Metoprolol Succinate (U.S. Pat. No. 5,081,154).
